(19) United States
(12) Patent Application Publication
(10) Pub. No.: US 2024/0315862 A1
(43) Pub. Date: Sep. 26, 2024

(54) ORTHOSIS FOR RANGE OF MOTION FOR AMPUTEE

(71) Applicant: BONUTTI RESEARCH, INC., Effingham, IL (US)

(72) Inventors: Glen A. Phillips, Effingham, IL (US); Bryan King, Alexandria, IN (US)

(21) Appl. No.: 18/615,712

(22) Filed: Mar. 25, 2024

Related U.S. Application Data
(60) Provisional application No. 63/492,030, filed on Mar. 24, 2023.

(51) Int. Cl. A61F 5/01

(52) U.S. Cl. CPC A61F 5/0123; A61F 5/013; A61F 2005/0172

(57) ABSTRACT

An orthosis is suitable for treating a joint of a subject that includes an amputated limb. The orthosis may be suitable for treating hinge joints (e.g., knee joint, elbow joint, and ankle joint) or ellipsoidal joints (e.g., wrist joint, finger joints, and toe joints) of the body. The orthosis includes a residual-limb cuff for coupling a residual limb to the orthosis. The residual-limb cuff includes a residual-limb liner. The residual-limb liner includes a liner body configured to surround the residual limb.

### CROSS-REFERENCE TO RELATED APPLICATION

The present application claims priority to U.S. Provisional Application No. 63/492,030, filed Mar. 24, 2023, the entirety of which is hereby incorporated by reference.

### FIELD OF THE DISCLOSURE

The present disclosure generally relates to an orthosis for treating a joint of a subject, and in particular, and orthosis for increasing range of motion of the joint of the subject with an amputated limp.

### BACKGROUND OF THE DISCLOSURE

In a joint of a body, its range of motion depends upon the anatomy and condition of that joint and on the particular genetics of each individual. Many joints primarily move either in flexion or extension, although some joints also are capable of rotational movement in varying degrees. Flexion is to bend the joint and extension is to straighten the joint; however, in the orthopedic convention some joints only flex. Some joints, such as the knee, may exhibit a slight internal or external rotation during flexion or extension. Other joints, such as the elbow or shoulder, not only flex and extend but also exhibit more rotational range of motion, which allows them to move in multiple planes. The elbow joint, for instance, is capable of supination and pronation, which is rotation of the hand about the longitudinal axis of the forearm placing the palm up or the palm down. Likewise, the shoulder is capable of a combination of movements, such as abduction, internal rotation, external rotation, flexion and extension.

When a joint is injured, either by trauma or by surgery, scar tissue can form or tissue can contract and consequently limit the range of motion of the joint. For example, adhesions can form between tissues and the muscle can contract itself with permanent muscle contracture or tissue hypertrophy such as capsular tissue or skin tissue. Lost range of motion may also result from trauma such as excessive temperature (e.g., thermal or chemical burns) or surgical trauma so that tissue planes which normally glide across each other may become adhered together to markedly restrict motion. The adhered tissues may result from chemical bonds, tissue hypertrophy, proteins such as Actin or Myosin in the tissue, or simply from bleeding and immobilization. It is often possible to mediate, and possibly even correct this condition by use of a range-of-motion (ROM) orthosis.

ROM orthoses are used during physical rehabilitative therapy to increase the range-of-motion of a body joint. Additionally, they also may be used for tissue transport, bone lengthening, stretching of skin or other tissue, tissue fascia, and the like. When used to treat a joint, the device typically is attached on body portions on opposite sides of the joint so that is can apply a force to move the joint in opposition to the contraction.

A number of different configurations and protocols may be used to increase the range of motion of a joint. For example, stress relaxation techniques may be used to apply variable forces to the joint or tissue while in a constant position. “Stress relaxation” is the reduction of forces, over time, in a material that is stretched and held at a constant length. Relaxation occurs because of the realignment of fibers and elongation of the material when the tissue is held at a fixed position over time. Treatment methods that use stress relaxation are serial casting and static splinting. One example of devices utilizing stress relaxation is the JAS EZ orthosis, Joint Active Systems, Inc., Effingham, IL.

Sequential application of stress relaxation techniques, also known as Static Progressive Stretch (“SPS”) uses the biomechanical principles of stress relaxation to restore range of motion (ROM) in joint contractures. SPS is the incremental application of stress relaxation—stretch to position to allow tissue forces to drop as tissues stretch, and then stretching the tissue further by moving the device to a new position—repeated application of constant displacement with variable force. In an SPS protocol, the patient is fitted with an orthosis about the joint. The orthosis is operated to stretch the joint until there is tissue/muscle resistance. The orthosis maintains the joint in this position for a set time period, for example five minutes, allowing for stress relaxation. The orthosis is then operated to incrementally increase the stretch in the tissue and again held in position for the set time period. The process of incrementally increasing the stretch in the tissue is continued, with the pattern being repeated for a maximum total session time, for example 30 minutes. The protocol can be progressed by increasing the time period, total treatment time, or with the addition of sessions per day. Additionally, the applied force may also be increased.

Another treatment protocol uses principles of creep to constantly apply a force over variable displacement. In other words, techniques and devices utilizing principles of creep involve continued deformation with the application of a fixed load. For tissue, the deformation and elongation are continuous but slow (requiring hours to days to obtain plastic deformation), and the material is kept under a constant state of stress. Treatment methods such as traction therapy and dynamic splinting are based on the properties of creep.

### SUMMARY

In one aspect, an orthosis for increasing range of motion of a body joint proximal to an amputated limb generally comprises: a proximal cuff configured to couple to a proximal body part proximal of the body joint; a residual-limb cuff configured to couple to residual limb distal of the body joint; and a drive mechanism operatively coupled to the proximal cuff and the residual-limb cuff and configured to drive selective movement of the residual-limb cuff relative to the proximal cuff. The residual-limb cuff includes a residual-limb liner. The residual-limb liner includes a liner body configured to surround the residual limb.

### BRIEF DESCRIPTION OF THE DRAWINGS

FIG. 1 is a front perspective of one embodiment of an orthosis configurable for use in treating a body joint in extension or flexion, the orthosis including a proximal cuff and a residual-limb cuff.

FIG. 2 is a front elevation of the orthosis.

FIG. 3 is a rear elevation of the orthosis.

FIG. 4 is a rear elevation of the orthosis with the cuffs removed.

FIG. 5 is a front elevation of the orthosis with the cuffs removed.

FIG. 6 is an exploded perspective of a dynamic force mechanism coupled to a fixed link and a bell crank of the orthosis.

FIG. 7 is a front perspective of a proximal limb cuff coupling of the orthosis of FIG. 1.

FIG. 8 is a front perspective of a residual-limb cuff coupling of the orthosis of FIG. 1.

FIG. 9A is a perspective the residual-limb liner secured to a residual-limb liner adapter.

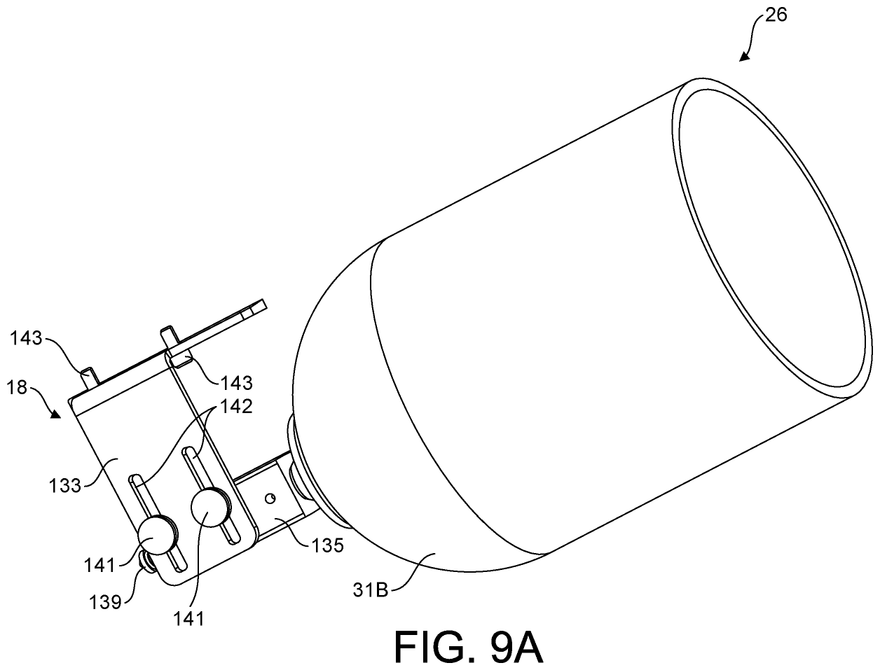

FIG. 9B is a is an exploded perspective the residual-limb liner removed from the residual-limb liner adapter.

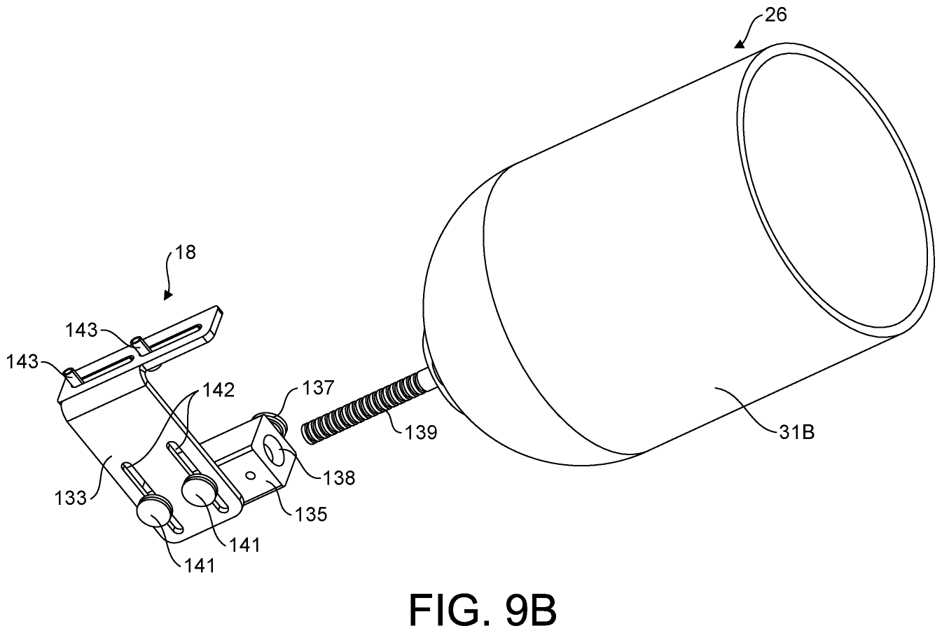

FIG. 10 is a partial cross section of the residual-limb liner and secured to a pin receiver of the residual-limb liner adapter.

FIG. 11 is a front elevation of another embodiment of an orthosis including the residual-limb cuff and the residual-limb liner adapter.

FIG. 12 is a front elevation of yet another embodiment of an orthosis including the residual-limb cuff and the residual-limb liner adapter.

Corresponding reference characters indicate corresponding parts throughout the drawings.

### DETAILED DESCRIPTION OF THE DISCLOSURE

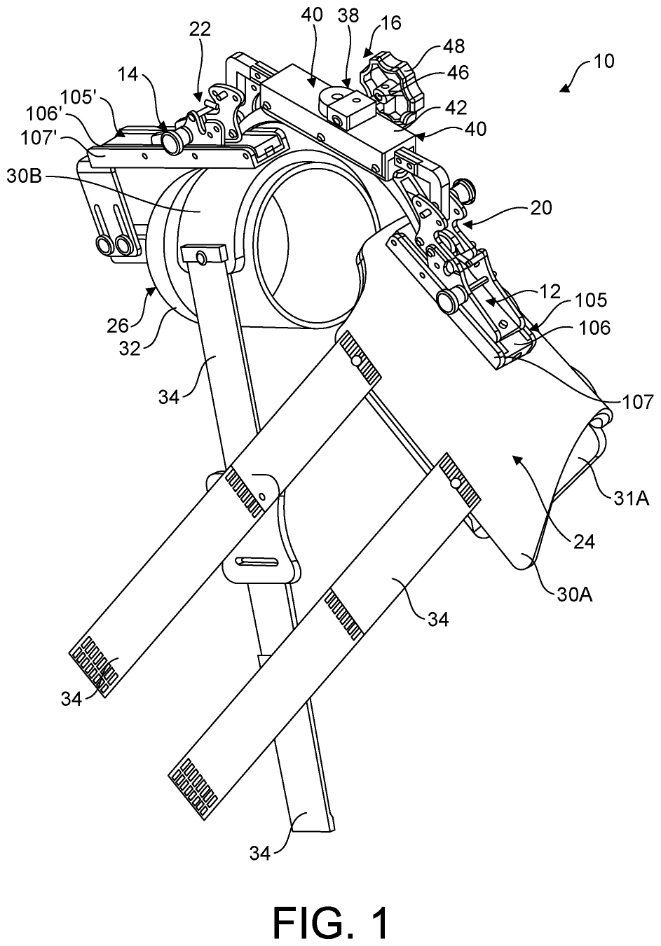

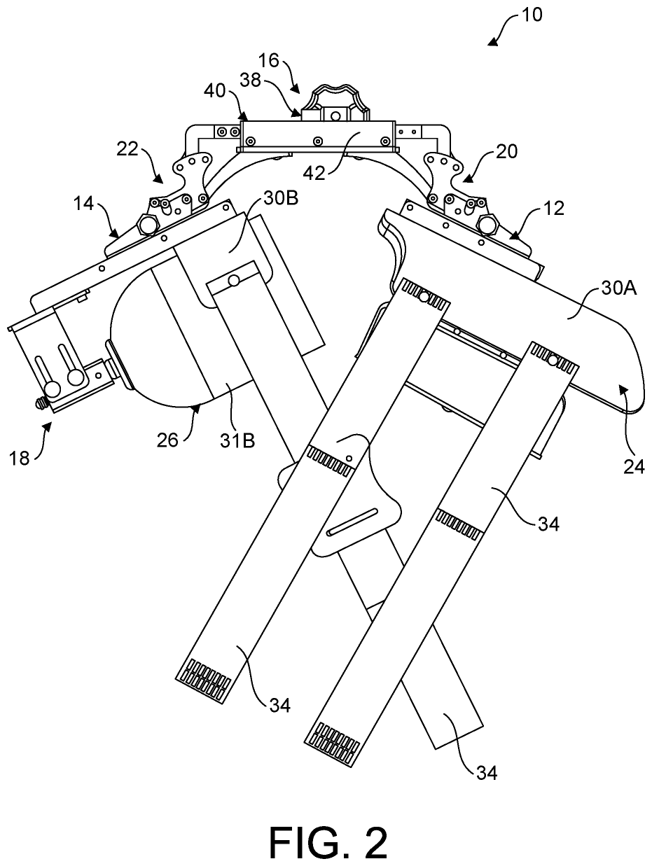

Referring to FIGS. 1-7, an orthosis for treating a joint of a subject that includes an amputated limb is generally indicated at reference numeral 10. The general structure of the orthosis illustrated in FIGS. 1-7 is suitable for treating hinge joints (e.g., knee joint, elbow joint, and ankle joint) or ellipsoidal joints (e.g., wrist joint, finger joints, and toe joints) of the body. For example, the knee joint may be treated for subjects with a below-the-knee amputation that removed a foot and part of the lower leg by cutting across the bones of the lower leg (tibia and fibula). Alternatively, the elbow joint may be treated for subjects with a below-the-elbow amputation that removed part of a lower arm by cutting across the bones of the lower arm (radius and ulna). It is understood that the illustrated embodiment of the orthosis is just one type of orthosis to which the teachings of the present disclosure may be incorporated. In particular, the teachings of the present disclosure are directed an orthosis configured for use with an amputated limb of a user.

The configuration of the orthosis 10 is suitable for increasing range of motion of a body joint in both extension and flexion, as explained in more detail below. Various teachings of the orthosis set forth herein are also suitable for orthoses for treating other joints, including but not limited to the shoulder joint, and the radioulnar joint. Thus, in other embodiments the teachings of the illustrated orthosis may be suitable for increasing range of motion of a body joint in adduction and/or abduction (e.g., the shoulder joint) or in pronation and/or supination (e.g., the radioulnar joint), among other joints. The illustrated embodiments are suitable for applying a dynamic stretch or load to a joint. It is understood that the embodiments may be modified to apply a static stretch or load to a joint, such as by omitting the dynamic force mechanisms, which are described below.

Referring to FIGS. 1-7, the illustrated orthosis 10 is configured as a dynamic stretch orthosis comprising first and second dynamic force mechanisms, generally indicated at 12, 14, respectively, for applying a dynamic stretch to respective first and second body portions on opposite sides of a body joint. As set forth above, the orthosis in general may be of other designs and constructions. An actuator mechanism, generally indicated at 16, is operatively connected to first and second linkage mechanism, generally indicated at 20, 22, respectively, for transmitting force to respective first and second dynamic mechanisms 12, 14 and loading the dynamic force mechanism during use, as will be explained in more detail below. Proximal and residual-limb cuffs, generally indicated at 24, 26, respectively (broadly, body portion securement members), are secured to the respective first and second dynamic force mechanisms 12, 14 for coupling the body portions to the first and second dynamic force mechanisms. As explained in more detail below, the residual-limb cuff 26 is coupled to the second dynamic force mechanism 14 on a distal end with a using a residual-limb cuff adapter 18. Each cuff 24, 26 may include a shell (e.g., plastic shell) 30A, 30B and at least one strap 34 secured to the plastic shell for fastening the body portion to the cuff. The strap(s) 34 may include a hook-and-loop fastener as is generally known in the art. The shells 30A, 30B may have inner padding or a lining 31A (padding for shell 30B not visible) secured thereto. The residual-limb cuff 26 further includes a residual-limb liner 32 has a liner body configured to surround or fully wrap around the residual limb, and includes an open end for receiving the residual limb and an opposite closed end. The liner body includes a soft, flexible sleeve (e.g., silicone sleeve) and a relatively rigid pin mount at the closed end of the liner body. The residual-limb liner 32 may be similar to or comprise a prosthetic liner used for prosthetic limbs of amputees. The shell 30B may be generally rigid relative to the residual-limb liner 32. The shell 30B may be generally U-shaped or arcuate for receiving the liner 32 and may not extend along the full length of the liner.

In one non-limiting example, for a lower extremity amputation the proximal cuff 24 may be configured for coupling to an upper leg portion of a subject, and the residual-limb cuff 26 may be configured for coupling to a residual limb portion of the lower leg of the subject to treat a knee joint of the subject. In another non-limiting example, for an upper extremity amputation the proximal cuff 24 may be configured for coupling to an upper arm portion of a subject, and the residual-limb cuff 26 may be configured for coupling to a residual limb portion of the lower arm of the subject to treat an elbow joint of the subject. In yet another non-limiting example, for a metacarpal amputation the proximal cuff 24 may be configured for coupling to a lower arm portion of a subject, and the residual-limb cuff 26 may be configured for coupling to a residual limb portion of a hand portion of the subject for treating a wrist joint of the subject. In another non-limiting example, for a syme amputation the proximal cuff 24 may be configured for coupling to a lower leg portion of a subject, and the residual-limb cuff 26 may be configured for coupling to a residual limb portion of a foot portion of the subject for treating an ankle joint of the subject. It is understood that the proximal and residual-limb cuffs 24, 26 may be configured for coupling to other body portions for treating other joints of the subject without departing from the scope of the present invention.

As will be understood through the following disclosure, the orthosis 10 may be used as a combination dynamic and static-progressive stretch orthosis. It is understood that in other embodiments the dynamic force mechanisms may be “locked out” thereby making the orthosis 10 suitable as a static stretch or static progressive stretch orthosis by utilizing the actuator mechanism 16 and/or linkage mechanism of the illustrated orthosis. In addition, it is understood that that in other embodiments the orthosis 10 may include the illustrated dynamic force mechanisms, while omitting the illustrated actuator mechanism and/or linkage mechanism making it a dynamic force mechanism, or utilizing a different type of actuator. The orthosis 10 may be of other types.

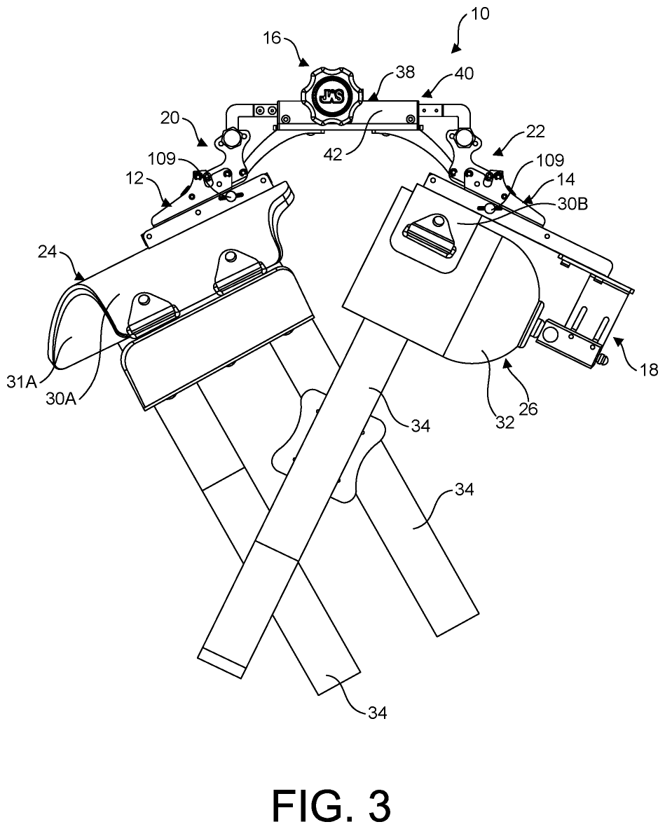

Referring to FIG. 3, the actuator mechanism 16 includes a drive assembly, generally indicated at 38, and a transmission assembly (e.g., a gear box), generally indicated at 40, operatively connected to the drive assembly. The actuator mechanism 16 may be substantially similar to an actuator mechanism described in U.S. Ser. No. 16/423,941, filed May 28, 2019, the specific disclosure of which is hereby incorporated by reference. Briefly, the transmission assembly 40 is contained within a transmission housing 42, and a portion of the drive assembly 38 extends outside the transmission housing. The drive assembly 38 includes a rotatable input shaft 46, a knob 48 accessible outside the transmission housing 42, and optionally a clutch mechanism (not shown), which operatively connects the knob to the input shaft to transmit torque from the knob to the input shaft. The knob 48 and input shaft 46 are rotatable about a common input. The knob 48 is configured to be grasped by a user (e.g., the subject) and rotated about the input axis to impart rotation of the input shaft 46 about the input axis. It is understood that the input shaft 46 may be operatively connected to a prime mover, such as a motor or engine, for rotating the input shaft, rather than a knob 48 or other components for manual operation of the orthosis 10. The drive assembly 38 may be of other configurations without departing from the scope of the present invention. The transmission assembly 40 includes a gearbox having meshed gears. The gearbox is coupled to the first and second linkage mechanisms 20, 22 to drive movement of the linkage mechanisms, as described below.

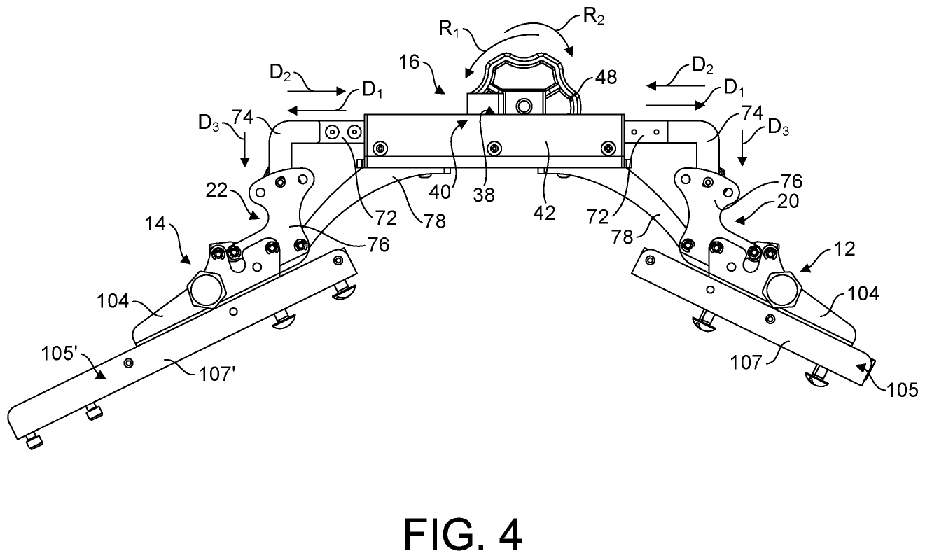

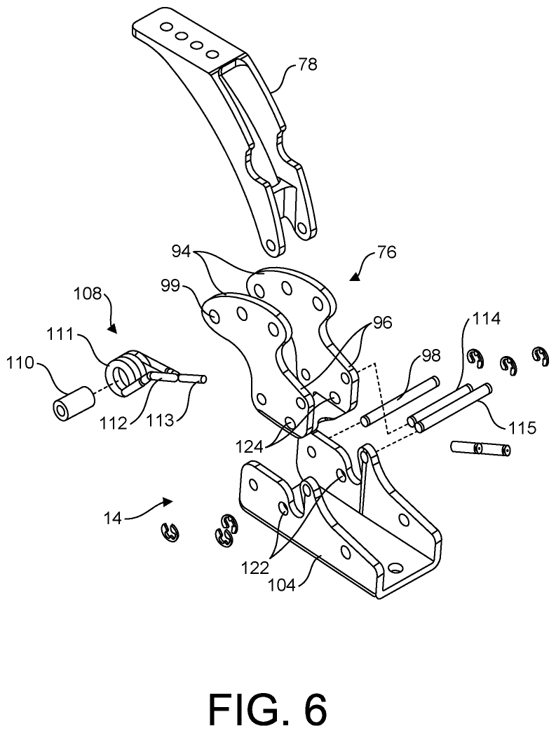

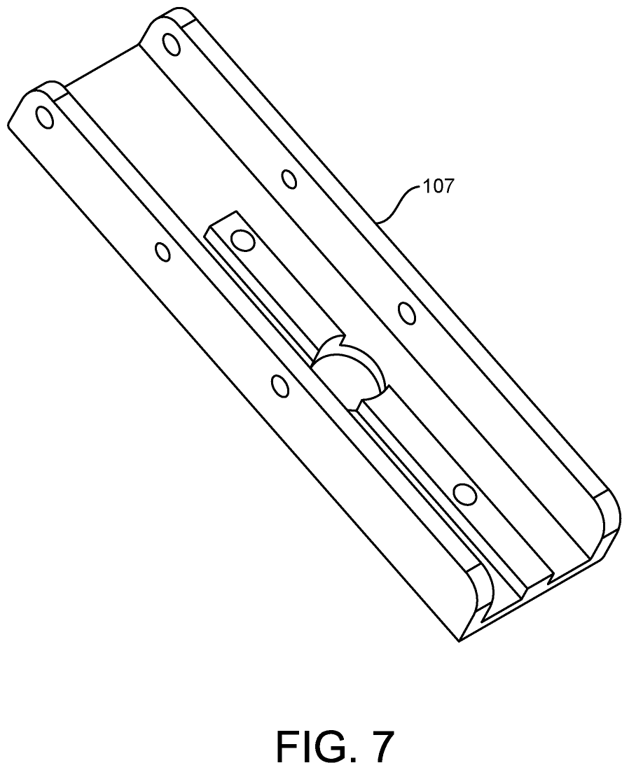

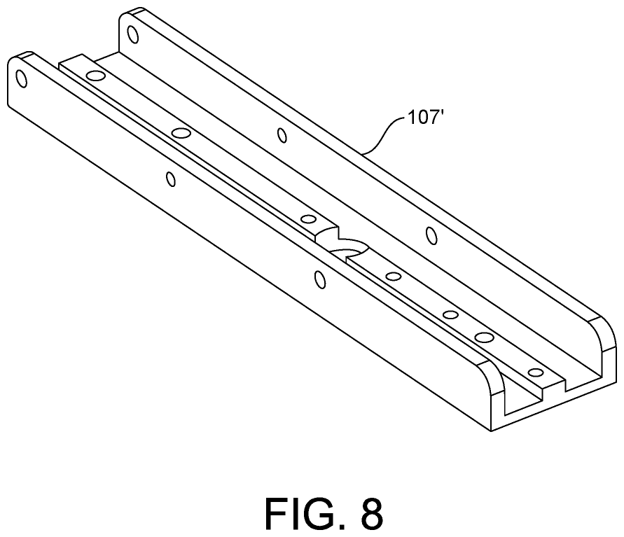

Referring to FIGS. 6-7, each of the first and second linkage mechanisms 20, 22 includes a sliding link 72, a yoke link 74, a bell crank, generally indicated at 76, and a fixed link 78. The first and second linkage mechanisms 20, 22 may be of similar construction, although dimensions of the components of the respective linkage mechanisms may be slightly different depending on the body joint to be treated. In the illustrated embodiment, the sliding link 72 of each of the first and second linkage mechanisms 20, 22 is operatively connected to (e.g., meshing engagement with) an output gear of the transmission assembly 40 to form a dual rack and pinion mechanism, whereby the sliding links are configured as racks and the output gear is configured as a pinion. The sliding links 72 are slidably received in the transmission housing 42 such that linear sets of teeth extending along the respective sliding links are in opposing relationship and the output gear (e.g., a pinion) is disposed between the linear sets of teeth. Rotation of the output gear about the output axis, as driven by rotation of the knob 48, imparts linear movement of the first and second sliding links 72 in opposite directions. As shown in FIG. 4, rotation of the knob 48 in a first direction RI about the input axis moves the sliding links 72 along linear paths in opposite first directions D1, away from one another. As shown in FIG. 8, rotation of the knob 48 in a second direction R2, opposite the first direction R1, about the input axis moves the sliding links 72 along linear paths in opposite second directions D2, toward one another. Accordingly, the illustrated actuator mechanism 16 is configured as a linear actuator mechanism which converts rotational movement (e.g., rotation of the knob 48) into linear movement of the first and second sliding links 72.

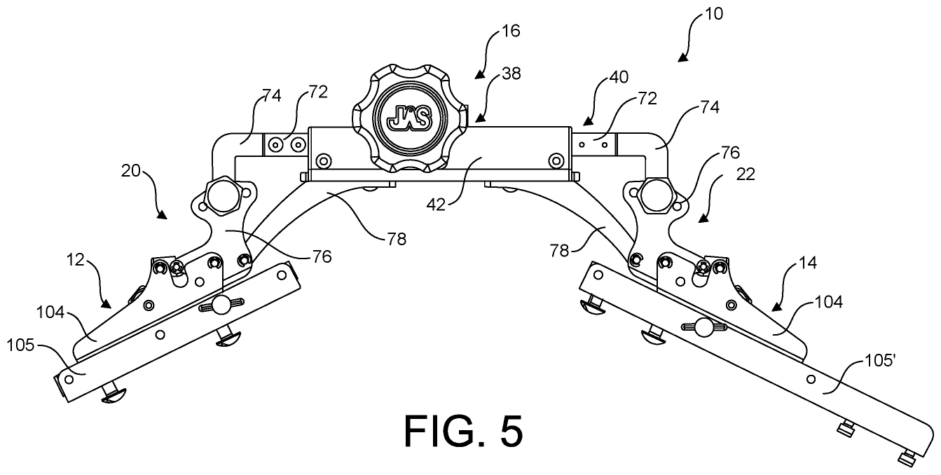

The first and second yoke links 74 are secured to ends of the respective first and second sliding links 72 that are outside the transmission housing 42. In the illustrated embodiment, the yoke links 74 are fastened (e.g., bolted) to the respective first and second sliding links 72, although it is understood that the yoke links may be integrally formed with the first and second sliding links. By making the yoke links 74 separate from the sliding links 72, yoke links with different sizes/configurations can be interchangeable on the orthosis 10 to accommodate different body joint sizes and/or different body joints. As shown best in FIG. 5, each of the yoke links 74 define a slot-shaped opening 90 having a length extending generally transverse (e.g., orthogonal) to the lengths and linear paths of the respective first and second sliding linkages.

Referring to FIG. 6, the first and second bell crank links 76 of the respective first and second linkage mechanisms 20, 22 each have a first crank arm or portion 94 (or first pair of arms or portions) operatively (i.e., slidingly) connected to the corresponding yoke link, and a second crank arm or portion 96 (or second pair of arms or portions) extending outward from the first crank arm. Yoke pins 97 are received in the slot-shaped openings 90 of the corresponding yoke links 74 and the first crank arms 94 to slidably secure terminal ends of the first crank arms 94 to the yoke links, thereby allowing sliding movement of the first crank arms relative to the corresponding yoke links. The yoke pins 97 are selectively removable and can be inserted into one of a plurality of pin openings 99 defined by the first crank arms 94 to change an initial or “zeroed out” angular position of the bell crank links 76. The first and second bell crank links 76 are rotatably (e.g., pivotably) attached to terminal ends of the respective first and second fixed links 78 generally at the second crank arms 96. In particular, fixed link pins 98 pivotably connect the first and second bell cranks 76 to the respective first and second fixed links 78 so that the bell crank links 76 are rotatable about pivot axes PA1. The other ends of the fixed links 78 are fixedly secured to an underside of the transmission housing 42, such as by fasteners 100 (e.g., screws), as shown in FIG. 3. In the illustrated embodiment, the locations of the fixed links 78 on the transmission housing 42 are adjustable to change a distance between the pivot axes of the first and second bell crank links 76 to accommodate joints and body portions of different sizes and/or different joints.

In operation, as shown in FIG. 4, rotation of the knob 48 in the first direction R1 imparts linear movement of the first and second sliding links 72 such that the yoke links 74 move away from one another to increase the distance between the yoke links. Moving the yoke links 74 away from one another imparts rotation of the first and second bell cranks 76 about the pivot axes in a flexion direction such that the second crank arms 96 pivot toward one another, and the first crank arms 94 slide along the slot-shaped openings 90 as shown by arrows D3. As the second crank arms 96 pivot toward one another in the flexion direction, an included angle between axes of the cuffs 24, 26 (and the second crank arms) decreases. Rotation of the knob 48 in the second direction imparts linear movement of the first and second sliding links 72 such that the yoke links 74 move toward one another to decrease the distance between the yoke links. As shown in FIG. 4, moving the yoke links 74 toward one another imparts rotation of the first and second bell cranks 76 about the pivot axes in an extension direction such that the second crank arms 96 pivot away from one another, and the first crank arms 94 slide along the slot-shaped openings 90. As the second crank arms 96 pivot away from one another in the extension direction, the included angle between axes of the cuffs 24, 26 (and the second crank arms) increases. Accordingly, the actuator mechanism 16 and the linkage mechanism are used to adjust an angular position of the first and second cuffs 24, 26 relative to one another to facilitate extension and flexion of the body joint. The intersection of the axes of the cuffs 24, 26 (i.e., the effective pivot point of the cuffs) moves as the cuffs are pivoted about the pivot axes.

As shown throughout the drawings, the first and second dynamic force mechanisms 12, 14 are operatively connected to the respective first and second bell cranks 76. In the illustrated embodiment, the dynamic force mechanisms 12, 14 are generally configured as levers, each comprising a lever arm 104 pivotably connected to the corresponding one of the bell cranks 76 by the pin 98 functioning as a fulcrum. In the illustrated embodiment, the pins 98 pivotably connect the bell cranks 76 to the corresponding fixed links 78 and the lever arms 104. Cuff couplings 105, 105′ are fixedly coupled to the lever arms 104. The cuffs 24, 26 are in turn coupled to the respective cuff couplings 105, 105′. In the illustrated embodiment, the cuff couplings 105, 105′ are configured to be selectively adjustable to adjust a distance between the cuffs 24, 26, so that the orthosis 10 is suitable for extension and flexion treatment. In general, each cuff coupling 105, 105′ includes a fixed block 106, 106′ attached to the corresponding lever 104, and a sliding block 107, 107′ secured to each cuff 24, 26 (such as by fasteners) and slidably coupled to the fixed block along a track. Each of the fixed block 106 and the sliding block 107 define openings that are alignable and configured to receive a removable pin 109 to releasably fix the longitudinal position of the sliding block 107 on the fixed block 106.

Referring to FIG. 6, resilient force elements 108 (e.g., torsion spring) apply forces to the respective lever arms 104 to pivot the lever arms about the pivot pins 98 and relative to the respective bell cranks 76 (more specifically, the second crank arms 96 of the bell cranks). In the illustrated embodiment, the force elements 108 are torsion springs mounted on a bushing 110 received on the pin 98. Torsion spring 108 includes a coiled body 111 surrounding the bushing 110 and upper and lower spring arms 112, 113 extending outward from the coiled body. Each bell crank 76 (e.g., the second crank arm 96) includes a spring-loading actuator 114. The illustrated spring-loading actuator 114 is in the form of a pin extending between opposing plates of the bell crank 76, although the spring-loading actuator may be of other structure in other embodiments. Each lever 104 includes a force-applying actuator 115. The illustrated force-applying actuator 115 is in the form of a pin extending between opposing plates of the lever 104, although the force-applying actuator may be of other structures in other embodiments. The torsion spring 108 applies a dynamic flexion force to the lever 104 (and therefore the cuffs 24, 26) when the lever 104 engages the upper spring arm 112 and the lower spring arm 113 engages the spring-loading actuator 114 so that the lower spring arm moves away from the upper spring arm to load the torsion spring. In this way, the loaded torsion spring 108 applies a dynamic force to the lever 104 in the direction of flexion, as shown by the arrow, about the pivot pin 98. The torsion spring 108 applies a dynamic extension force to the lever 104 (and therefore the cuffs 24, 26) when the spring-loading actuator 114 engages the upper spring arm 112 and the lower spring arm 113 engages the force-applying actuator 115 so that the upper spring arm is moved away from the lower spring arm to load the torsion spring. In this way, the loaded torsion spring 108 applies a dynamic force to the lever 104 in the direction of extension, as shown by the arrow, about the pivot pin 98. This operation is described in U.S. Ser. No. 16/423,941, filed May 28, 2019, the specific disclosure of which is hereby incorporated by reference.

The proximal cuff 24 is secured to the corresponding sliding block 107 using fasteners (e.g., screws or bolts), for example, extending through the outer shell 30A and secured in the sliding block. This type of coupling is described in U.S. Ser. No. 16/423,941, filed May 28, 2019, the specific disclosure of which is hereby incorporated by reference. The residual-limb cuff 26 is secured to the coupling 105′ (more specifically, the sliding block 107′) by i) fasteners (e.g., screws or bolts) extending through the outer shell 30B and into the sliding block 107, and ii) the residual-limb liner adapter 18 secured to the liner 32 and to the sliding block 107. In the illustrated embodiment, the residual-limb liner 32 may not be directly attached to the cuff shell 30B when the strap is not secured around the liner to allow adjustable of the liner relative to the cuff shell for residual limbs of different shapes, sizes, and lengths, as explained in more detail below. The residual-limb cuff 26 may be secured to the coupling 105′ in other ways, in other embodiments, including secured using the fasteners extending through the outer shell 30B or the residual-limb liner adapter 18.

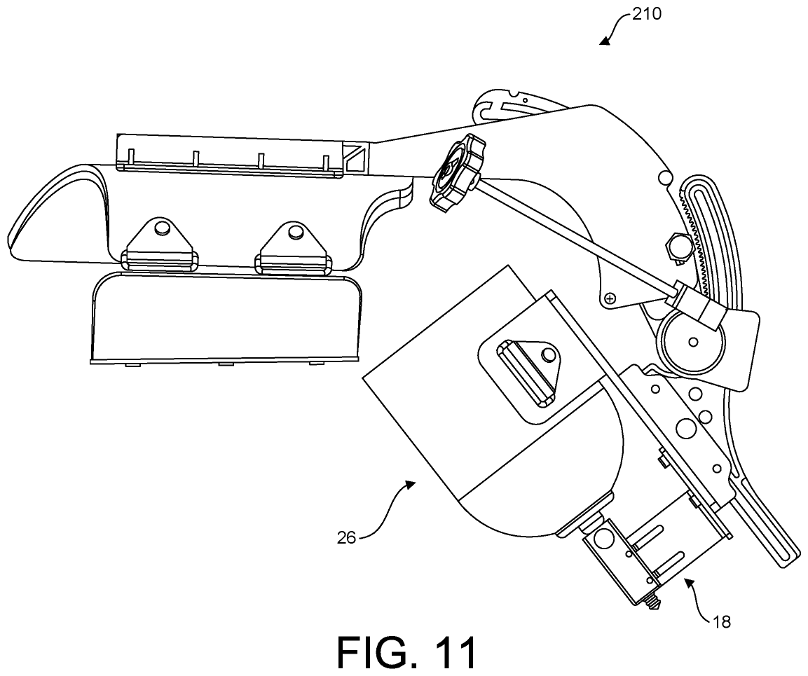

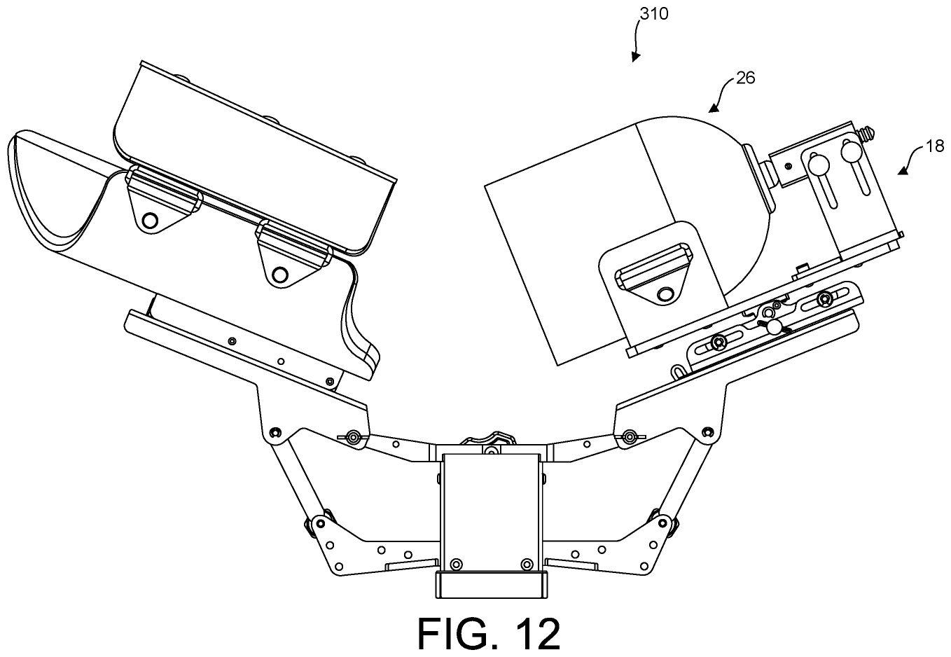

Referring to FIGS. 11A-12, the residual-limb liner adapter 18 includes a mounting bracket 133 secured to the coupling 105′, such as by fasteners 143, and a pin receiver 135 secured to the mounting bracket. The illustrated mounting bracket 133 has an L-shaped body, with a first portion receiving the fasteners 143 therethrough to secure the first portion to the coupling 105′, and a second portion receiving fasteners 141 therethrough to secure the pin receiver 135 to the second portion. The second portion defines slots 142 extending lengthwise of the second portion and configured to receive the fasteners 141 therethrough. The fasteners 141 (e.g., thumbscrews) may be selectively loosened to enable the pin receiver 135 to be moved toward or away from the coupling 105′ and tightened to lock the position of the pin receiver. The adapter 18 may be selectively adjustable in other ways, and in particular, other ways to allow for selective adjustment of the pin receiver 135 relative to the coupling 105′, both toward and away from the coupling 105′.

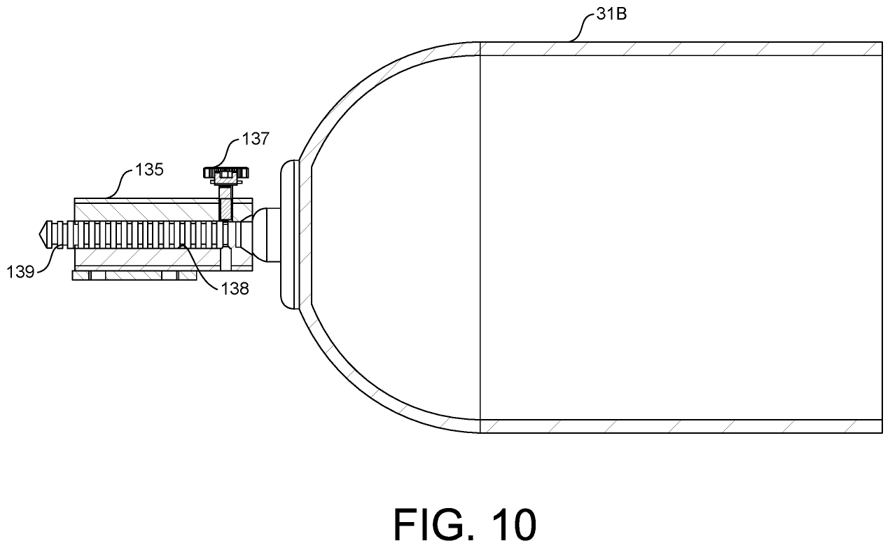

The illustrated pin receiver 135 comprises a block or body defining an opening 138 configured to receive a liner pin 139 extending axially from the closed end (e.g., pin mount) of the residual-limb liner 32. The liner pin 139 is fixedly secured to the closed end of the residual-limb liner 32, such as by adhesive, welding, fasteners, or in other ways. The illustrated pin 139 defines a plurality of notches or grooved spaced apart along the length of the pin. As shown in FIG. 10, a set screw 137 is threadable through the body of the pin receiver 135 and into the opening 138 for reception in a selective one of the notches of the pin 139. In this way, the pin 139 and residual-limb liner 32 are selectively movable relative to the pin receiver (and the residual-limb liner adapter 18) to adjust the distance between the residual-limb liner 32 and the residual-limb liner adapter 18. The residual-limb liner 32 may be coupled to the adapter 18 in other ways, and in particular, other ways to allow for selective adjustment of the residual-limb liner relative to the adapter.

As can be seen from the above disclosure, the residual-limb cuff 26 enables use of the orthosis 10 on an amputated limb. Moreover, the residual-limb liner 32 of the illustrated residual-limb cuff 26 is adjustable to enable use with amputees having different residual limb shapes, sizes and lengths. In one aspect, the residual-limb liner 32 may be an off-the-shelf prosthetic liner or bespoke prosthetic liner for the amputee that includes the pin 139. The amputee can use the residual-limb liner 32 with the orthosis by donning the residual-limb liner and then inserting the pin 139 into the pin receiver 135. The amputee or other person can adjust the position of the residual-limb liner 32 to align the liner properly on the residual limb of the amputee so that the joint is properly positioned on the orthosis. In particular, the liner 32 can be easily adjusted toward or away from the coupling 105′ in a first direction, and can be easily adjusted toward and away from the proximal cuff 24 to properly don the orthosis on the amputee's limb. Then, the strap 34 can be tightened and secured around the residual-limb liner 32, thereby securing the residual limb to the orthosis 10.

As disclosed above, the orthosis 10 is suitable for increasing range of motion of a body joint in extension or flexion. In an exemplary method of use, a first body portion is secured to the proximal cuff 24 and a second body portion, i.e., a residual limb or stump, on an opposite side of a joint, for example, is secured to the residual-limb cuff 26. As a non-limiting example, in the embodiment illustrated in FIG. 1, an upper leg or upper arm portion can be secured to the proximal cuff 24 and a lower residual limb can be secured to the residual-limb cuff 26 for treating a knee or elbow joint in extension. In the illustrated embodiment, the first body portion is secured to the cuff 24 by placing the first body portion in the cuff and using the straps and the hook and loop fasteners on the straps. The residual limb or stump is secured to the cuff 26 by inserting the residual limb in the residual-limb liner 32 (e.g., prosthetic liner), and then inserting the pin 139 into the pin receiver 135. The position of the residual-limb liner can be adjusted in two different planes that are orthogonal to one another. After proper positioning, the residual-limb liner 32 is locked by tightening the fasteners 141 and fastener 137. Alternatively, the user may insert the residual limb in the residual-limb liner 32 after proper positioning and locking of the liner.

With the body portions secured to the respective cuffs 24, 26, the subject extends the body joint to a desired, initial position in extension or flexion, such as a position recommended by a healthcare professional and/or to a maximum initial position in extension or flexion to which the subject can move the body joint. In another example, the desired initial rotational position of the bell cranks 76 may be set before donning the orthosis 10 by applying force to the bell cranks using one's hands, for example. With the bell cranks 76 in the desired initial angular position, the cuffs 24, 26 may be secured to the respective body portions to position the body joint in the desired, initial position in extension or flexion. In one example, the initial position of the bell cranks 76 may be facilitated by inserting the pins 97 in the desired openings 99 of the bell cranks to position the bell cranks in the desired rotational position.

With the body portions secured to the orthosis 10 and the body joint in the desired, initial position in extension or flexion, the knob 48 is rotated to impart rotation of the bell cranks 76 in the extension or flexion direction, depending on the desired treatment. At some point in the range of motion in extension or flexion of the body joint (e.g., at the initial extension or flexion position of the body joint), rotation of the bell cranks 76 in the extension or flexion direction does not impart further extension or flexion of the body joint because the stiffness of the body joint overcomes the biasing force of the springs 108.

With respect to flexion treatment using the orthosis 10, further rotation of the bell cranks 76 in the flexion direction moves the second crank arms 96 of the bell cranks toward the lever arms 104 and the cuffs 24, 26 (e.g., relative pivoting of the bell cranks and the levers), as the lever arms and the cuffs stay with the body portions and do not move. As the second crank arms 96 of the bell cranks 76 pivot toward the lever arms 104 about the pin 98, the spring-loading actuators 114 move the lower spring arms 113 away from the upper spring arms 112 to elastically deform and load the springs 108. Elastic deformations of the springs 108 produce a dynamic force on the force-applying actuators 115 via the upper spring arms 112, thereby producing a biasing force against the lever arms 104 in a direction away from corresponding second crank arms 96 of the bell cranks 76. Further pivoting of the bell cranks 76 by turning the knob 48 decreases the angular distance between the second cranks arms 96 and the corresponding lever arms 104, thereby increasing the dynamic force of the spring 108 imparted on the body portions in the flexion direction. The bell cranks 76 are pivoted to a suitable treatment position in which the biasing forces of the springs 108 are constantly applied to both sides of the body joint in the flexion direction. The application of this biasing force utilizes the principles of creep to continuously stretch the joint tissue during a set time period (e.g., 4-8 hours), thereby maintaining, decreasing, or preventing a relaxation of the tissue.

With respect to extension treatment using the orthosis 10, further rotation of the bell cranks 76 in the extension direction moves the second crank arms 96 of the bell cranks away from the lever arms 104 and the cuffs 24, 26 (e.g., relative pivoting of the bell cranks and the levers), as the lever arms and the cuffs stay with the body portions and do not move. As the second crank arms 96 of the bell cranks 76 pivot away from the lever arms 104 about the pin 98, the spring-loading actuators 114 move the upper spring arms 112 away from the lower spring arms 113 to elastically deform and load the springs 108. Elastic deformations of the springs 108 produce a dynamic force on the force-applying actuators 115 via the upper spring arms 112, thereby producing a biasing force against the lever arms 104 in a direction toward the corresponding second crank arms 96 of the bell cranks 76 (as indicated by force DEF). Further pivoting of the bell cranks 76 by turning the knob 48 increases the angular distance between the second cranks arms 96 and the corresponding lever arms 104, thereby increasing the dynamic force of the spring 108 imparted on the body portions in the extension direction. The bell cranks 76 are pivoted to a suitable treatment position in which the biasing forces of the springs 108 are constantly applied to both sides of the body joint in the extension direction. The application of this biasing force utilizes the principles of creep to continuously stretch the joint tissue during a set time period (e.g., 4-8 hours), thereby maintaining, decreasing, or preventing a relaxation of the tissue.

In the illustrated embodiment, the dynamic force mechanisms 12, 14 pin 139 may be individually locked-out to inhibit dynamic force being applied to the levers 104 and therefore the cuffs 24, 26. In one example, a lock-out pin 120 is insertable into aligned openings 122, 124 in the respective levers 104 and bell cranks 76. Once received in the aligned openings 122, 124, each lock-out pin 120 fixedly couples the respective lever 104 to the corresponding bell crank 76 so that the lever moves with the bell crank rather than being movable relative to one another. In this way, the spring 108 is inhibited from applying a dynamic load to the lever 104, thereby configuring the orthosis as a static-progressive orthosis only.

Referring to FIG. 11, another embodiment of an orthosis is generally indicated at reference numeral 210. This orthosis 210 is of a different type than the first orthosis 10, however, this orthosis includes the same residual-limb cuff 26 and adapter 18 described above. Yet another embodiment including the residual-limb cuff 26 and adapter 18 is indicated at reference numeral 310 in FIG. 12. As can be seen, the residual-limb cuff 26 and adapter 18 can be used on various types of orthoses.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. An orthosis for increasing range of motion of a body joint proximal to an amputated limb, the orthosis comprising:
a proximal cuff configured to couple to a proximal body part proximal of the body joint;
a residual-limb cuff configured to couple to residual limb distal of the body joint; and
a drive mechanism operatively coupled to the proximal cuff and the residual-limb cuff and configured to drive selective movement of the residual-limb cuff relative to the proximal cuff,
wherein the residual-limb cuff includes a residual-limb liner, the residual-limb liner including a liner body configured to surround the residual limb.

2. The orthosis set forth in claim 1, wherein the liner body has an open end for receiving the residual limb.

3. The orthosis set forth in claim 2, wherein the liner body a closed end opposite the open end.

4. The orthosis set forth in claim 3, wherein the residual-limb cuff includes a residual-limb shell secured to the residual-limb liner.

5. The orthosis set forth in claim 4, wherein the residual-limb shell is arcuate and the liner body is received in the residual-limb shell.

6. The orthosis set forth in claim 5, wherein the residual-limb cuff includes a strap coupled to the residual-limb shell, the strap configured to wrap around the liner body to secure the residual limb in the residual-limb liner.

7. The orthosis set forth in claim 3, wherein the residual-limb liner includes pin extending from the closed end of the liner body.

8. The orthosis set forth in claim 7, wherein the liner body includes a soft, flexible sleeve and a relatively rigid pin mount at the closed end of the liner body, the pin being secured to the pin mount.

9. The orthosis set forth in claim 7, further comprising a residual-limb adapter operatively coupling the residual-limb liner to the drive assembly.

10. The orthosis set forth in claim 9, wherein the residual-limb adapter includes a mounting bracket and a pin receiver secured to the mounting bracket, wherein the mounting bracket operatively couples the pin receiver to the drive assembly, wherein the liner pin is secured to the pin receiver.

11. The orthosis set forth in claim 10, wherein the liner pin is adjustably secured to the pin receiver such that the liner pin and the residual-limb liner are selectively movable relative to the pin receiver to adjust the distance between the residual-limb liner and the residual-limb liner adapter.

12. The orthosis set forth in claim 11, wherein pin receiver defines an opening configured to receive the liner pin, the residual-limb adapter including a set screw threaded through the pin receiver and into the opening, wherein the set screw is configured to enable the liner pin to be selectively i) fixedly secured in the opening of the pin receiver and ii) free to move axially in the opening of the pin receiver.

13. The orthosis set forth in claim 11, wherein the pin receiver is adjustably secured to the mounting bracket such that the pin receiver is selectively movable relative to the mounting bracket.

14. The orthosis set forth in claim 13, wherein the pin receiver is selectively movable relative to the mounting bracket in a direction transverse to an axis of the liner pin.

15. The orthosis set forth in claim 14, wherein mounting bracket defines a slot, the residual-limb adapter including a set screw received in the slot and threaded into the pin receiver and into the opening, wherein the set screw is configured to enable the pin receiver to be selectively i) fixedly secured to the mounting bracket and ii) free to move along the mounting bracket to adjust a position of the pin receiver on the mounting bracket.

16. The orthosis set forth in claim 15, wherein the slot comprises a pair of parallel slots, and the set screw comprises a pair of set screws.

17. The orthosis set forth in claim 1, wherein the orthosis is configured to increase range of motion of a knee joint, wherein the proximal cuff is configured to couple to an upper leg and the residual-limb cuff is configured to couple to a residual limb portion of a corresponding lower leg.

18. The orthosis set forth in claim 1, wherein the orthosis is configured to increase range of motion of an elbow joint, wherein the proximal cuff is configured to couple to an upper arm and the residual-limb cuff is configured to couple to a residual limb portion of a corresponding lower arm.

19. The orthosis set forth in claim 1, wherein the orthosis is configured to increase range of motion of a wrist joint, wherein the proximal cuff is configured to couple to a lower arm and the residual-limb cuff is configured to couple to a residual limb portion of a corresponding hand.

20. The orthosis set forth in claim 1, wherein the orthosis is configured to increase range of motion of an ankle joint, wherein the proximal cuff is configured to couple to a lower leg and the residual-limb cuff is configured to couple to a residual limb portion of a corresponding foot.
